(19) United States
(12) Patent Application Publication
(10) Pub. No.: US 2012/0071505 A1
(43) Pub. Date: Mar. 22, 2012

(54) SUBSTITUTED PYRIDINE DERIVATIVES, PHARMACEUTICAL COMPOSITIONS, AND METHODS OF USE TO TREAT OXIDATIVE STRESS

(75) Inventors: Bapu Gaddam, High Point, NC (US); Dharma Rao Polisetti, High Point, NC (US); Matthew J. Kostura, Hillsborough, NC (US); Mustafa Guzel, Jamestown, NC (US); Samuel Victory, Oak Ridge, NC (US)

(73) Assignee: HIGH POINT PHARMACEUTICALS, LLC, High Point, NC (US)

(21) Appl. No.: 13/278,515

(22) Filed: Oct. 21, 2011

Related U.S. Application Data
(63) Continuation of application No. PCT/US2010/044508, filed on Aug. 5, 2010.
(60) Provisional application No. 61/234,498, filed on Aug. 17, 2009.

(51) Int. Cl. A61K 31/47; C07D 215/24; A61P 9/00; A61P 9/10; A61P 9/12; A61P 3/10; A61P 13/12; A61P 25/28; A61P 25/16; A61P 25/00; A61P 11/06; A61P 11/00; A61P 17/00; A61P 27/02; A61P 27/12; A61P 35/00; C07D 215/54

(52) U.S. Cl. 514/312; 546/156; 546/155

(57) ABSTRACT

Substituted pyridine derivatives, methods of their preparation, pharmaceutical compositions comprising a substituted pyridine derivative, and methods of use in treating inflammation are provided. The substituted pyridine derivatives may control of the activity or the amount or both the activity and the amount of heme-oxygenase.

### CROSS REFERENCE TO RELATED APPLICATIONS

The present application is a continuation of international application No. PCT/US2010/044508, filed Aug. 5, 2010, which claims the benefit of priority to U.S. Provisional Patent Application No. 61/234,498, filed Aug. 17, 2009.

### BACKGROUND OF THE INVENTION

1. Field of the Invention

This invention relates substituted pyridine derivatives that may be useful for the control of the inflammatory response. In particular, this invention relates to control of the activity or the amount or both the activity and the amount of heme-oxygenase.

2. Description of Related Art

Cellular damages due to oxidative stress caused by reactive oxygen species (ROS) has been demonstrated to be involved in the onset or progression of various chronic diseases (for example, cardiovascular disease including arteriosclerosis and hypertension; diabetes and diabetic related complications such as glomerular nephropathy; cerebral nerve degenerative diseases such as Alzheimer's disease, Parkinson's disease, ALS (amyotrophic lateral sclerosis) and multiple sclerosis; asthma, chronic obstructive pulmonary disease, skin diseases, eye diseases, and cancer). Enhancing the capability in protecting from oxidative stress may be useful in either preventing these diseases, delaying their progress, or delaying their onset. Further, with the varied etiology associated with this diverse set of diseases, a general strategy to mitigate oxidative stress would be beneficial.

The basic biochemistry of a cell generates ROS including superoxide anion, hydroxyl anion, nitric oxide, peroxynitrite and hydrogen peroxide. All of these products subserve critical cellular signaling needs but have deleterious consequences if overproduced or left unchecked. Many disease conditions induce persistent levels of ROS that are associated with the establishment of chronic pathophysiologic changes seen within a variety of tissues. These complications, in and of themselves, may be the primary drivers of disease morbidity and mortality.

Under normal physiological conditions, production of ROS are counterbalanced by a well defined and conserved set of cellular pathways that respond to, limit and repair the damage due to ROS. This adaptive set of genes, called the phase II system, encode enzymes that degrade ROS directly (e.g. superoxide dismutase and catalase) as well as increase levels of a cell's endogenous antioxidant molecules including glutathione and bilirubin. Examples of known phase II enzymes include glutathione S-transferase (GST), NAD(P)H:quinone oxidoreductase 1 (NQO1), glutamyl-cysteinyl ligase (GCL), heme oxygenase 1 (HMOX1), and thioredoxin reductase 1 (TXNRD1). A common sequence called antioxidant responsive element (ARE) is present in a promoter of each gene of these phase II enzymes, and its expression is induced by the transcription factor Nrf2 (NF-E2 related factor 2).

Of the Phase II enzyme system, HMOX1 has been found to be a key component. HMOX1 role is to metabolize hemin into bilirubin, carbon monoxide and free iron as first step of the two step process to catabolize of heme. The first, and rate limiting reaction, is the production of biliverdin and carbon monoxide from heme by HMOX1. The second step is the production of bilirubin from biliverdin by biliverdin reductase. Both bilirubin and carbon monoxide have been shown to scavenge ROS and to have potent anti-oxidant and anti-inflammatory activity. Agents that induce production of HMOX1 have been shown to have beneficial activity in models of diabetes, cardiovascular disease, hypertension and pulmonary function.

HMOX1 is found in liver, kidney, spleen and skin, and has also been localized to specific cell types, notably fibroblasts and macrophages. HMOX1 exists in at least three isoforms, one constitutive and the other two inducible. Heme, heavy metal ions (e.g., tin, gold, platinum and mercury), transition metal ions (e.g., iron, cobalt, chromium and nickel), and electrophiles (e.g., natural products such as sulforophan and curcumin) can all induce production of HMOX1. Induction of HMOX1 and other phase II genes are controlled by a number of transcription factors that are responsive to heavy metals, hemin and electrophiles. The transcription factors Nrf2, Bach1 and Maf are particularly important in this process. In addition, there are cofactors and regulatory molecules that are important in regulating Phase II gene induction. These include Keap1, an adapter molecule targeting Nrf2 for ubiquitination, and two mitochondrial proteins, DJ-1 and frataxin (FXN) that serve to augment Nrf2 activation in the presence of electrophiles. HMOX1 is also induced as part of a generalized stress response to stimuli such as thermal shock, oxidative stress and cytokines such as interleukin-1 (IL-1), tumor necrosis factor and IL-6. The stress response is seen as beneficial in that it results in protection of vulnerable cell enzymes from inactivation.

### BRIEF SUMMARY OF INVENTION

This invention provides substituted pyridine derivatives and pharmaceutical compositions which reduce oxidative stress and/or inflammation. In an embodiment, the present invention provides compounds of Formula (I) and pharmaceutically acceptable salts thereof as depicted below. In another embodiment, the present invention provides methods of preparation of compounds of Formula (I) and pharmaceutically acceptable salts thereof. In another embodiment, the present invention provides pharmaceutical compositions comprising a compound of Formula (I) or a pharmaceutically acceptable salt thereof. In another embodiment, the present invention provides methods of treatment comprising: administering to a subject a compound of Formula (I) or a pharmaceutically acceptable salt thereof.

The compounds of Formula (I) and pharmaceutically acceptable salts thereof are useful as agents that induce production of and/or increase activity of HMOX1 and thus may be useful to treat various chronic diseases that are associated at least in part with oxidative stress such as, but not limited to, cardiovascular disease including arteriosclerosis and hypertension; diabetes and diabetic related complications such as glomerular nephropathy; cerebral nerve degenerative diseases such as Alzheimers disease, Parkinson's disease, ALS (amyotrophic lateral sclerosis) and multiple sclerosis; asthma; chronic obstructive pulmonary disease; skin diseases; eye diseases including macular degeneration, cataracts, light retinopathy, and retinopathy of prematurity; and cancer.

### BRIEF DESCRIPTION OF DRAWINGS

Not applicable

### DETAILED DESCRIPTION

### Definitions

As used herein, “treat” or “treating” or “treatment” can refer to one or more of: delaying the progress of a disease, disorder, or condition; controlling a disease, disorder, or condition; ameliorating one or more symptoms characteristic of a disease, disorder, or condition; or delaying the recurrence of a disease, disorder, or condition, or characteristic symptoms thereof, depending on the nature of the disease, disorder, or condition and its characteristic symptoms.

As used herein, “subject” refers to any mammal such as, but not limited to, humans, horses, cows, sheep, pigs, mice, rats, dogs, cats, and primates such as chimpanzees, gorillas, and rhesus monkeys. In an embodiment, the “subject” is a human. In another embodiment, the “subject” is a human who exhibits one or more symptoms characteristic of a disease, disorder, or condition. The term “subject” does not require one to have any particular status with respect to a hospital, clinic, or research facility (e.g., as an admitted patient, a study participant, or the like).

As used herein, the term “compound” includes free acids, free bases, and salts thereof. Thus, phrases such as “the compound of embodiment 1” or “the compound of claim 1” are intended to refer to any free acids, free bases, and salts thereof that are encompassed by embodiment 1 or claim 1.

As used herein, the phrase “substituted pyridine derivatives” refers to the substituted pyridine derivatives of Formula (I) and pharmaceutically acceptable salts thereof.

In the compounds of Formula (I) or pharmaceutically acceptable salts thereof, the various functional groups represented should be understood to have a point of attachment at the functional group having the hyphen or asterisk. In other words, in the case of —C₁₋₆ alkylaryl, it should be understood that the point of attachment is the alkyl group; an example would be benzyl. In the case of a group such as —C(O)—NH—C₁₋₆ alkylaryl, the point of attachment is the carbonyl carbon. Further, in the case of variable having a group with two points of attachment such as —NHC(O)—, the group is inserted into the variable in a left to right manner.

As used herein the term “alkyl” refers to a straight or branched chain hydrocarbon having one to twelve carbon atoms, which may be substituted as herein further described, with multiple degrees of substitution being allowed. Examples of “alkyl” as used herein include, but are not limited to, methyl, ethyl, propyl, isopropyl, isobutyl, n-butyl, tert-butyl, isopentyl, and n-pentyl.

As used throughout this specification, the number of atoms, such as carbon atoms in an alkyl group, for example, will be represented by the phrase “Cₓ-C_{y} alkyl,” or “C_{x-y} alkyl,” which refer to an alkyl group, as herein defined, containing from x to y, inclusive, carbon atoms. Similar terminology will apply for other terms and ranges as well.

As used herein the term “halogen” refers to fluorine, chlorine, bromine, or iodine.

As used herein the term “haloalkyl” refers to an alkyl group, as defined herein, that is substituted with at least one halogen. Examples of branched or straight chained “haloalkyl” groups as used herein include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups such as —CF₃.

When any variable occurs more than one time in any one constituent (e.g., R⁵⁰), or multiple constituents, its definition on each occurrence is independent at every other occurrence.

As used herein, the term “optionally” means that the subsequently described event(s) may or may not occur.

As used herein, the term “direct bond”, where part of a structural variable specification, refers to the direct joining of the substituents flanking (preceding and succeeding) the variable taken as a “direct bond”. Where two or more consecutive variables are specified each as a “direct bond”, those substituents flanking (preceding and succeeding) those two or more consecutive specified “direct bonds” are directly joined.

As used herein, the term “substituted” refers to substitution of one or more hydrogens of the designated moiety with the named substituent or substituents, multiple degrees of substitution being allowed unless otherwise stated, provided that the substitution results in a stable or chemically feasible compound. A stable compound or chemically feasible compound is one in which the chemical structure is not substantially altered when kept at a temperature from about −80° C. to about +40° C., in the absence of moisture or other chemically reactive conditions, for at least a week, or a compound which maintains its integrity long enough to be useful for therapeutic administration to a patient.

As used herein, the phrase “one or more substituents” refers to a number of substituents that equals from one to the maximum number of substituents possible based on the number of available bonding sites, provided that the above conditions of stability and chemical feasibility are met.

As used herein, “a subject” includes mammalian subjects such as, but not limited to, humans. In an embodiment, a subject is a human. In another embodiment, a subject is one who suffers from one or more of the aforesaid diseases, disease states, or conditions. In another embodiment, a subject is a human who suffers from one or more of the aforesaid diseases, disease states, or conditions.

Embodiments of the present invention comprises substituted pyridine derivatives, pharmaceutical compositions comprising substituted pyridine derivatives, method of making substituted pyridine derivatives, methods of making pharmaceutical compositions comprising substituted pyridine derivatives, and methods of use thereof.

In a first aspect, the present invention provides substituted pyridine derivatives that induce production of HMOX1 and thus may be useful to treat various diseases associated at least in part with oxidative stress.

In a first embodiment, the present invention provides a compound of Formula (I):

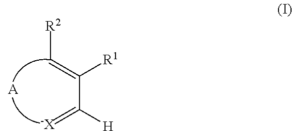

wherein

### A is

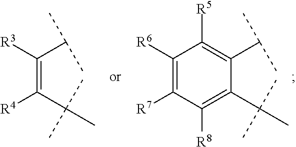

### X is N or N—O;

R¹ is the group -D¹-L¹-R¹¹, wherein
D¹ is selected from the group consisting of: direct bond, —C(O)—, —CO₂—, —NH—C(O)—, —S—, —S(O)—, —SO₂—, —CH═CH—CO₂—, and —C≡C—CH₂—,
L¹ is selected from the group consisting of: direct bond and C₁₋₆ alkylene, and
R¹¹ is selected from the group consisting of: hydrogen, C₁₋₆ alkyl, cyclopentyl, cyclohexyl, —O—C₁₋₆ alkyl, tetrahydrofuran-2-yl, tetrahydrofuran-3-yl, and phenyl, wherein the cyclopentyl, cyclohexyl, tetrahydrofuanyl, and phenyl groups are optionally substituted with one or more substituents independently selected from the group consisting of: C₁₋₆ alkyl, C₁₋₆ haloalkyl, —O—C₁₋₆alkyl, —O—C₁₋₆ haloalkyl, and halogen,
wherein when D¹ and L¹ are direct bonds, then R¹¹ is a phenyl group;
   R² is the group -D²-L²-R¹², wherein
D² is selected from the group consisting of: —S—, —SO₂—, and —NH—,
L² is selected from the group consisting of: direct bond and C₁₋₆ alkylene, and
R¹¹ is selected from the group consisting of: C₁₋₆ alkyl, cyclopentyl, cyclohexyl, —O—C₁₋₆ alkyl, furan-2-yl, furan-3-yl, tetrahydrofuran-2-yl, tetrahydrofuran-3-yl, pyridine-2-yl, and phenyl, wherein the cyclopentyl, cyclohexyl, furanyl, tetrahydrofuanyl, pyridine, and phenyl groups are optionally substituted with one or more substituents independently selected from the group consisting of: C₁₋₆ alkyl, C₁₋₆ haloalkyl, —O—C₁₋₆alkyl, —O—C₁₋₆ haloalkyl, and halogen;
   R³, R⁴, R⁵, R⁶, R⁷, and R⁸ are independently selected from the group consisting of:
hydrogen, —O—C₁₋₆ alkyl, and —O—C₁₋₆ halo alkyl;
   or a pharmaceutically acceptable salt thereof.

### Embodiment 2

A compound of Formula (I) or a pharmaceutically acceptable salt thereof according to embodiment 1, wherein A

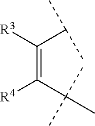

is so as to form the ring

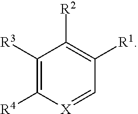

### Embodiment 3

A compound of Formula (I) or a pharmaceutically acceptable salt thereof according to embodiment 2, wherein R³ and R⁴ are hydrogen.

### Embodiment 4

A compound of Formula (I) or a pharmaceutically acceptable salt thereof according to embodiment 1, wherein A is

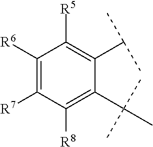

so as to form the ring

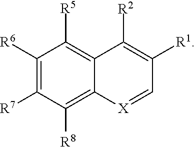

### Embodiment 5

A compound of Formula (I) or a pharmaceutically acceptable salt thereof according to embodiment 4, wherein R⁵ and R⁷ are hydrogen, and R⁶, and R⁸ are independently selected from the group consisting of: hydrogen, —O—C₁₋₆ alkyl, and —O—C₁₋₆ halo alkyl.

### Embodiment 6

A compound of Formula (I) or a pharmaceutically acceptable salt thereof according to embodiment 5, wherein R⁵ and R⁷ are hydrogen, and R⁶, and R⁸ are independently selected from the group consisting of: hydrogen, —O-methyl, and —O-halomethyl.

### Embodiment 7

A compound of Formula (I) or a pharmaceutically acceptable salt thereof according to embodiment 6, wherein R⁵, R⁶, and R⁷ are hydrogen, and R⁸ are independently selected from the group consisting of: hydrogen, —O-methyl, and —O-halomethyl.

### Embodiment 8

A compound of Formula (I) or a pharmaceutically acceptable salt thereof according to any one of embodiments 1 to 7, wherein X is N.

### Embodiment 9

A compound of Formula (I) or a pharmaceutically acceptable salt thereof according to any one of embodiments 1 to 7, wherein X is N—O.

### Embodiment 10

A compound of Formula (I) or a pharmaceutically acceptable salt thereof according to any one of the previous embodiments, wherein R¹ is the group -D¹-L¹-R¹¹, wherein
D¹ is selected from the group consisting of: direct bond, —C(O)—, —CO₂—, —NH—C(O)—, —S—, —S(O)—, —SO₂—, —CH═CH—CO₂—, and —C≡C—CH₂—,
L¹ is selected from the group consisting of: direct bond and C₁₋₆ alkylene, and
R¹¹ is selected from the group consisting of: C₁₋₆ alkyl, cyclopentyl, cyclohexyl, —O—C₁₋₆ alkyl, tetrahydrofuran-2-yl, tetrahydrofuran-3-yl, and phenyl, wherein the cyclopentyl, cyclohexyl, tetrahydrofuanyl, and phenyl groups are optionally substituted with one or more substituents independently selected from the group consisting of: C₁₋₆ alkyl, C₁₋₆ haloalkyl, —O—C₁₋₆alkyl, —O—C₁₋₆ haloalkyl, and halogen,
wherein when D¹ and L¹ are direct bonds R¹¹ is a phenyl group;

### Embodiment 11

A compound of Formula (I) or a pharmaceutically acceptable salt thereof according to embodiment 10, wherein D¹ is —CO₂—.

### Embodiment 12

A compound of Formula (I) or a pharmaceutically acceptable salt thereof according to embodiment 10, wherein D¹ is selected from the group consisting of: —C(O)—, —NH—C(O)—, —S—, —S(O)—, and —SO₂—.

### Embodiment 13

A compound of Formula (I) or a pharmaceutically acceptable salt thereof according to embodiment 10, wherein D¹ is selected from the group consisting of: —CH═CH—CO₂—, and —C≡C—CH₂—.

### Embodiment 14

A compound of Formula (I) or a pharmaceutically acceptable salt thereof according to embodiment 10, wherein R¹ is the group -D¹-L¹-R¹¹, and D¹ is a direct bond, L¹ is a direct bond, and R¹¹ is phenyl, wherein the phenyl group is optionally substituted with one or more substituents independently selected from the group consisting of: C₁₋₆ alkyl, C₁₋₆ haloalkyl, —O—C₁₋₆alkyl, —O—C₁₋₆ haloalkyl, and halogen.

### Embodiment 15

A compound of Formula (I) or a pharmaceutically acceptable salt thereof according to any one of embodiments 10 to 13, wherein L¹ is C₁₋₆ alkylene group.

### Embodiment 16

A compound of Formula (I) or a pharmaceutically acceptable salt thereof according to any one of embodiments 10 to 13 and 15, wherein R¹¹ is a —O—C₁₋₆ alkyl group.

### Embodiment 17

A compound of Formula (I) or a pharmaceutically acceptable salt thereof according to any one of the previous embodiments, wherein R² is the group -D²-L²-R¹², wherein
D² is selected from the group consisting of: —S—, —SO₂—, and —NH—,
L² is selected from the group consisting of: direct bond and C₁₋₆ alkylene, and
R¹² is selected from the group consisting of: C₁₋₆ alkyl, cyclopentyl, cyclohexyl, —O—C₁₋₆ alkyl, furan-2-yl, furan-3-yl, tetrahydrofuran-2-yl, tetrahydrofuran-3-yl, pyridine-2-yl, and phenyl, wherein the cyclopentyl, cyclohexyl, furanyl, tetrahydrofuanyl, pyridine, and phenyl groups are optionally substituted with one or more substituents independently selected from the group consisting of: C₁₋₆ alkyl, C₁₋₆ haloalkyl, —O—C₁₋₆alkyl, —O—C₁₋₆ haloalkyl, and halogen.

### Embodiment 18

A compound of Formula (I) or a pharmaceutically acceptable salt thereof according to embodiment 17, wherein D² is —SO₂—.

### Embodiment 19

A compound of Formula (I) or a pharmaceutically acceptable salt thereof according to embodiment 17, wherein D² is —NH—.

### Embodiment 20

A compound of Formula (I) or a pharmaceutically acceptable salt thereof according to any one of embodiments 17 to 19 wherein L² is a direct bond.

### Embodiment 21

A compound of Formula (I) or a pharmaceutically acceptable salt thereof according to any one of embodiments 17 to 19, wherein L² is a C₁₋₃ alkylene.

### Embodiment 22

A compound of Formula (I) or a pharmaceutically acceptable salt thereof according to any one of embodiments 17 to 21, wherein R¹² is selected from the group consisting of: furan-2-yl, furan-3-yl, tetrahydrofuran-2-yl, and tetrahydrofuran-3-yl, wherein the furanyl and tetrahydrofuanyl groups are optionally substituted with one or more substituents independently selected from the group consisting of: C₁₋₆ alkyl.

### Embodiment 23

A compound of Formula (I) or a pharmaceutically acceptable salt thereof according to embodiment 22, wherein R¹² is selected from the group consisting of: furan-3-yl and tetrahydrofuran-3-yl, wherein the furanyl and tetrahydrofuanyl groups are optionally substituted with one or more substituents independently selected from the group consisting of: C₁₋₆ alkyl.

### Embodiment 24

A compound of Formula (I) or a pharmaceutically acceptable salt thereof according to embodiment 1, wherein

A is

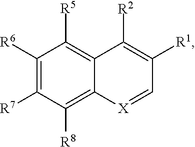

wherein X is N;

R¹ is the group -D¹-L¹-R¹¹, wherein
D¹ is —CO₂—,
L¹ is selected from the group consisting of: direct bond and C₁₋₆ alkylene, and
R¹¹ is selected from the group consisting of: C₁₋₆ alkyl, and —O—C₁₋₆ alkyl, wherein when L¹ is a direct bond R¹¹ is a C₁₋₆ alkyl group;

R² is the group -D²-L²-R¹², wherein
D² is —SO₂—,
L² is selected from the group consisting of: direct bond and —CH₂—, and
R¹¹ is selected from the group consisting of: furan-3-yl, and tetrahydrofuran-3-yl, wherein the furanyl and tetrahydrofuanyl groups are optionally substituted with one or more substituents independently selected from the group consisting of: C₁₋₆ alkyl,

R⁵, R⁶, R⁷, and R⁸ are independently selected from the group consisting of:
hydrogen, —O—C₁₋₆ alkyl, and —O—C₁₋₆ halo alkyl;

or a pharmaceutically acceptable salt thereof.

Included within the scope of the present invention are all stereoisomers, geometric isomers and tautomeric forms of the compounds of Formula (I), including compounds exhibiting more than one type of isomerism, and mixtures of one or more thereof. Also included within the scope of the present invention are stereoisomeric mixtures of compounds having Formula (I), or a diastereomerically enriched or diastereomerically pure isomer of a compound of Formula (I), or an enantiomerically enriched or enantiomerically pure isomer of a compound of Formula (I). The present invention includes all pharmaceutically acceptable isotopically-labelled compounds of Formula (I) wherein one or more atoms are replaced by atoms having the same atomic number, but an atomic mass or mass number different from the atomic mass or mass number usually found in nature. Examples of isotopes that may be suitable for inclusion in the compounds of the invention include isotopes of hydrogen, carbon, chlorine, fluorine, iodine, nitrogen, oxygen, phosphorus, and sulfur.

The routes in the Examples illustrate methods of synthesizing compounds of Formula (I). The skilled person will appreciate that the compounds of the invention could be made by methods other than those specifically described herein, by adaptation of the methods described herein and/or adaptation thereof, for example by methods known in the art.

Examples of compounds of Formula (I) or pharmaceutically acceptable salts thereof having useful biological activity are listed the Examples section and in Table 1. The ability of compounds of Formula (I) or pharmaceutically acceptable salts thereof to increase levels or activity of HMOX-1 was established using the Biological Assay described below.

TABLE 1

| | | Fold Change of |
|---|---|---|
| | | HMOX1 mRNA |
| | | expression at |
| Ex | | 3.75 μM |
| 1 | 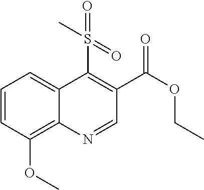 | 3.4 |
| 2 | 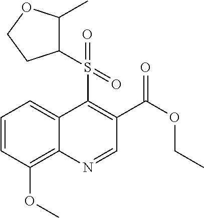 | 48.6 |
| 3 | 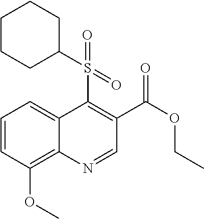 | 6.7 (at 3 μM) |
| 4 | 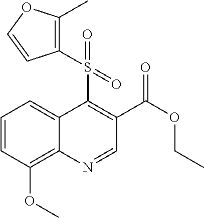 | 5.8 |
| 5 | 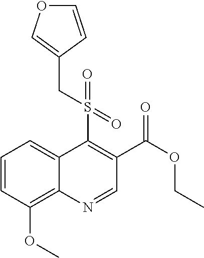 | 2.0 (at 10 μM) |
| 6 | 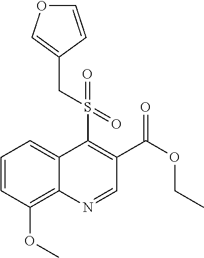 | 64.7 |
| 7 | 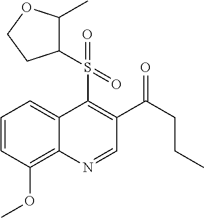 | 2.8 (at 10 μM) |
| 8 | 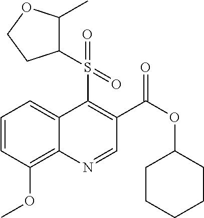 | 4.5 (at 3 μM) |
| 9 | 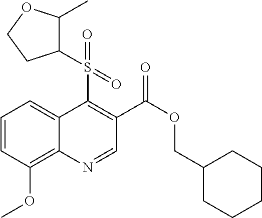 | 2 (at 3 μM) |
| 10 | 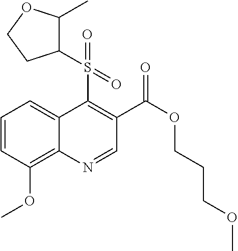 | 28.5 |
| 11 | 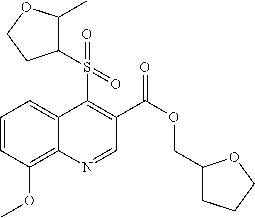 | 2.7 (at 3 μM) |
| 12 | 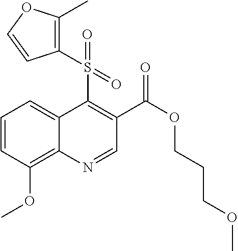 | 15.3 |
| 13 | 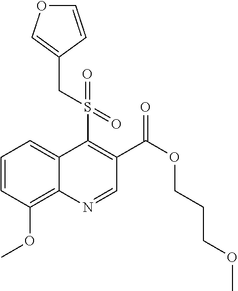 | 17.2 |
| 14 | 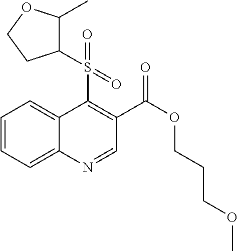 | 52.4 |
| 15 | 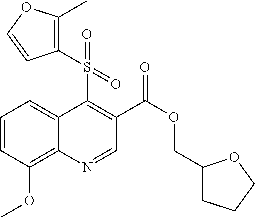 | 4.4 |
| 16 | 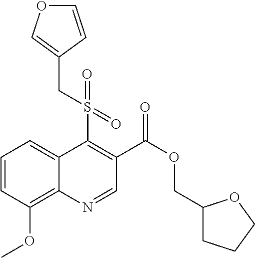 | 2.3 |
| 17 | 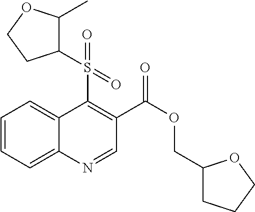 | 7.8 |
| 18 | 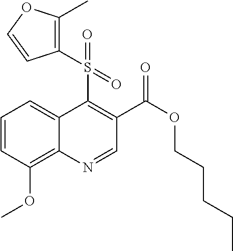 | 10.2 |
| 19 | 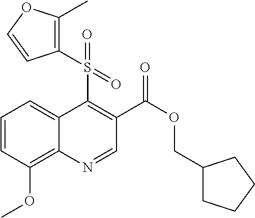 | 12.6 |
| 20 | 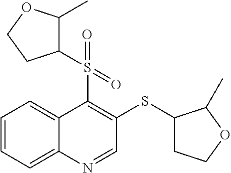 | 25.9 |
| 21 | 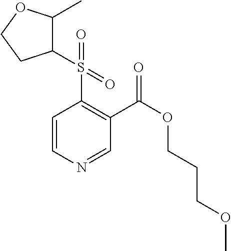 | 6.0 (at 10 μM) |
| 22 | 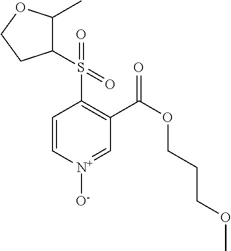 | 5.6 (at 10 μM) |
| 23 | 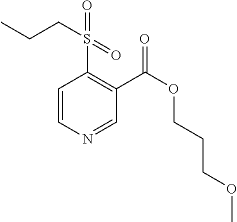 | 4.2 |
| 24 | 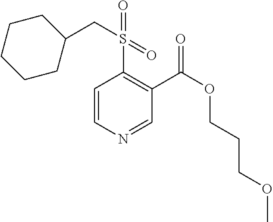 | 5.3 |
| 25 | 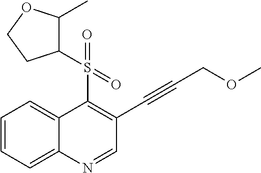 | 13 |
| 26 | 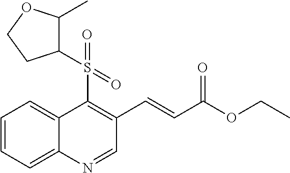 | 13.2 |
| 27 | 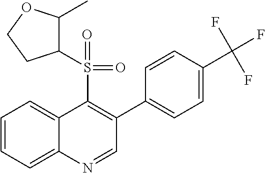 | 27.5 (at 2.5 μM) |
| 28 | 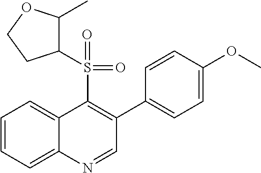 | 12.7 |
| 29 | 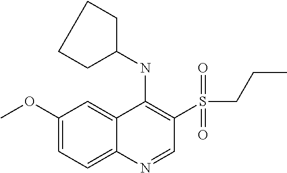 | 4.0 (at 3 μM) |
| 30 | 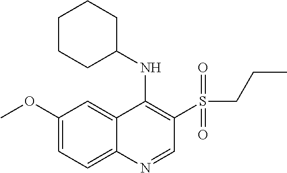 | 4.0 (at 3 μM) |
| 31 | 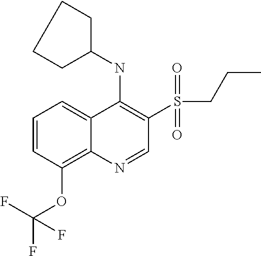 | 4.0 (at 3 μM) |
| 32 | 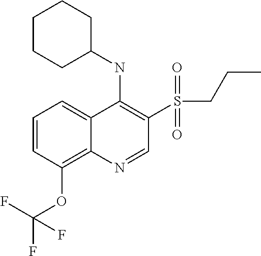 | 9.8 |

Compounds that increase levels or activity of HMOX1 are potentially useful in treating diseases or conditions that may be associated at least in part with oxidative stress such as, but not limited to, cardiovascular disease including arteriosclerosis and hypertension; diabetes and diabetic related complications such as glomerular nephropathy; cerebral nerve degenerative diseases such as Alzheimers disease, Parkinson's disease, ALS (amyotrophic lateral sclerosis) and multiple sclerosis; asthma; chronic obstructive pulmonary disease; skin diseases; eye diseases including macular degeneration, cataracts, light retinopathy, and retinopathy of prematurity; and cancer.

The compounds of Formula (I) or pharmaceutically acceptable salts thereof may therefore be useful in the treatment of one or more of these diseases.

In another aspect, the present invention comprises a pharmaceutical composition comprising the compound of Formula (I) or a pharmaceutically acceptable salt thereof. In an embodiment, the pharmaceutical composition further comprises a pharmaceutically acceptable carrier, excipient, diluent, or a mixture thereof.

In an embodiment, the pharmaceutical compositions containing a compound of Formula (I) or a pharmaceutically acceptable salt thereof may be in a form suitable for oral use, for example, as tablets, troches, lozenges, aqueous, or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, to form osmotic therapeutic tablets for controlled release.

In another embodiment, formulations for oral use may also be presented as hard gelatin capsules where the active ingredient is mixed with an inert solid diluent, for example, calcium carbonate, calcium phosphate or kaolin, or a soft gelatin capsules wherein the active ingredient is mixed with water or an oil medium, for example peanut oil, liquid paraffin, or olive oil.

In another embodiment, the composition may comprise an aqueous suspension. Aqueous suspensions may contain the active compounds in an admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more coloring agents, one or more flavoring agents, and one or more sweetening agents, such as sucrose or saccharin.

Also, oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavoring, and coloring agents may also be present.

The pharmaceutical compositions of the invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavoring agents.

In another embodiment, the pharmaceutical compositions of the present invention may comprise a syrup or elixir. Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The pharmaceutical compositions of the present invention may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

In an embodiment, for topical use, creams, ointments, jellies, solutions of suspensions, etc., containing the compounds of the invention may be employed. For the purpose of this application, topical applications shall include mouth washes and gargles.

In an embodiment, the compounds of Formula (I) and pharmaceutically acceptable salts thereof may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

Pharmaceutically-acceptable salts of compounds of Formula (I), where a basic or acidic group is present in the structure, are also included within the scope of the invention. The term “pharmaceutically acceptable salts” refers to salts of the compounds of this invention which are not biologically or otherwise undesirable and are generally prepared by reacting the free base with a suitable organic or inorganic acid or by reacting the acid with a suitable organic or inorganic base. Representative salts include the following salts: Acetate, Benzenesulfonate, Benzoate, Bicarbonate, Bisulfate, Bitartrate, Borate, Bromide, Calcium Edetate, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrocloride, Hydroxynaphthoate, Iodide, Isethionate, Lactate, Lactobionate, Laurate, Malate, Maleate, Mandelate, Mesylate, Methylbromide, Methylnitrate, Methylsulfate, Monopotassium Maleate, Mucate, Napsylate, Nitrate, N-methylglucamine, Oxalate, Pamoate (Embonate), Palmitate, Pantothenate, Phosphate/diphosphate, Polygalacturonate, Potassium, Salicylate, Sodium, Stearate, Subacetate, Succinate, Tannate, Tartrate, Teoclate, Tosylate, Triethiodide, Trimethylammonium and Valerate. When an acidic substituent is present, such as —COOH, there can be formed the ammonium, morpholinium, sodium, potassium, barium, calcium salt, and the like, for use as the dosage form. When a basic group is present, such as amino or a basic heteroaryl radical, such as pyridyl, an acidic salt, such as hydrochloride, hydrobromide, phosphate, sulfate, trifluoroacetate, trichloroacetate, acetate, oxalate, maleate, pyruvate, malonate, succinate, citrate, tartarate, fumarate, mandelate, benzoate, cinnamate, methanesulfonate, ethanesulfonate, picrate and the like, and include acids related to the pharmaceutically-acceptable salts listed in the Journal of Pharmaceutical Science, 66, 2 (1977) p. 1-19.

Other salts which are not pharmaceutically acceptable may be useful in the preparation of compounds of the invention and these form a further aspect of the invention.

Thus, in a further embodiment, there is provided a pharmaceutical composition comprising a compound of Formula (I) or a pharmaceutically acceptable salt thereof, and one or more pharmaceutically acceptable carriers, excipients, or diluents.

One or more kinds of medically effective active ingredients other than an active ingredient according to the present invention can be further admixed or otherwise combined in one dosage form with a compound of Formula (I) or a pharmaceutically acceptable salt thereof. Further, upon administering a compound of Formula (I) or a pharmaceutically acceptable salt thereof, one or more kinds of active ingredient other than a compound of Formula (I) or a pharmaceutically acceptable salt thereof may be administered. Examples of such other active ingredients include, but are not limited to, antioxidants, detoxification agents, and anti-inflammatory agents.

Examples of the Nrf2 activators include sulforaphane, avicins, 15dPGJ₂, xanthohumol, curcumin, carnosol, zerumbone, isothiocyanate, α-lipoic acid, oltipraz (4-methyl-5-[2-pyrazinyl]-1,2-dithiole-3-thione), 1,2-dithiole-3-thione, and 2,3-butyl-4-hydroxyanisole. Examples of the antioxidants include vitamin C, vitamin E, carotenoids, retinolds, polyphenols, flavonoids, lignan, selenium, butylated hydroxyanisole, ethylene diamine tetra-acetate, calcium disodium, acetylcysteine, probucol, and tempo. Examples of the detoxification agents include dimethyl caprol, glutathione, acetylcysteine, methionine, sodium hydrogen carbonate, deferoxamine mesylate, calcium disodium edetate, trientine hydrochloride, penicillamine, and pharmaceutical charcoal. The anti-inflammatory agents include steroidal anti-inflammatory agents and non-steroidal anti-inflammatory agents. Examples of the steroidal anti-inflammatory agents include cortisone acetate, hydrocortisone, paramethasone acetate, prednisolone, prednisolone, methylprednine, dexamethasone, triamcinolone, and betamethasone. Examples of the non-steroidal anti-inflammatory agents include salicylic acid non-steroidal anti-inflammatory agents such as aspirin, difiunisal, aspirin+ascorbic acid, and aspirin dialuminate; aryl acid non-steroidal anti-inflammatory agents such as diclofenac sodium, sulindac, fenbufen, indomethacin, indomethacin farnesyl, acemetacin, proglumetacin maleate, anfenac sodium, nabmeton, mofezolac, and etodorag; fenamic acid non-steroidal anti-inflammatory agents such as mefenamic acid, flufenamic acid aluminum, tolfenamic acid, and floctafenine; propionic acid non-steroidal anti-inflammatory agents such as ibuprofen, flurbiprofen, ketoprofen, naproxen, pranoprofen, fenoprofen calcium, thiaprofen, oxaprozin, loxoprofen sodium, alminoprofen, and zaltoprofen; oxicam non-steroldal anti-inflammatory agents such as piroxicam, ampiroxicam, tenoxicam, lornoxicam, and meloxicam; and basic non-steroidal anti-inflammatory agents such as tiaramide hydrochloride, epirizole, and emorfazone.

### Methods of Use

A compound of Formula (I) or pharmaceutically acceptable salt thereof, or a pharmaceutical composition comprising a compound of Formula (I) or pharmaceutically acceptable salt thereof may be used for the treatment of a disease or condition which is treatable by activation of transcription factor Nrf2, by increasing activity and/or amount of HMOX1, or by reducing amounts of ROS in a subject.

Examples of a disease or condition which may be treatable by activation of transcription factor Nrf2, by increasing activity and/or amount of HMOX1, or by reducing amounts of ROS in a subject include cerebral nerve degenerative diseases, eye diseases, skin diseases, asthma, cancer, arteriosclerosis and diseases or conditions related thereto. Examples of the cerebral nerve degenerative diseases include Alzheimer's disease, Parkinson's disease, and amyotrophic lateral sclerosis. Further, examples of the eye diseases include age-related macular degeneration, cataracts, light retinopathy, and retinopathy of prematurity. Specific examples of the chronic inflammatory diseases include vasculitis, pulmonary bronchitis, rheumatoid arthritis, osteoarthritis, hepatitis, pancreatitis, dermatitis, esophagitis, ulcerative colitis, Crohn's disease, and conjunctivitis. Further examples of conditions that may be treatable include thrombosis and diseases of the kidney. Thus the present invention provides a method of treatment comprising administering to a subject a compound of Formula (I) or a pharmaceutically acceptable salt thereof so as to treat one of the diseases or conditions listed above.

In another embodiment, the present invention provides a method of treating inflammation comprising administering to a subject a compound of Formula (I) or a pharmaceutically acceptable salt thereof so as to increase the activity or amount of HMOX1 in a subject.

This invention also provides a method of treatment of chronic inflammation comprising administering to a subject a compound of Formula (I) or a pharmaceutically acceptable salt thereof so as to increase the activity or amount of HMOX1 in a subject.

This invention further provides a method of treatment of a disease selected from rheumatoid arthritis, a chronic inflammatory bowel disease, multiple sclerosis, asthma, airways inflammatory disease, tendonitis and chronic inflammation in the brain, comprising administering to a subject a compound of Formula (I) or a pharmaceutically acceptable salt thereof so as to increase the activity or amount of HMOX1 in a subject.

According to one aspect of the invention, inflammation is treated by administration of a compound that induces heme-oxygenase or stimulates or increases the activity of heme-oxygenase. The treatment can be systemic or targeted, for example targeted to an inducible heme-oxygenase found in monocytes and macrophages in the human body. This treatment induces heme-oxygenase production and/or stimulates its activity; specific medical applications include the treatment of chronic inflammatory diseases for example, rheumatoid arthritis, the treatment of hypersensitivity reactions such as in asthma, and, the treatment of injury, atherosclerosis and infarction.

In another embodiment, the present invention provides a method of treatment of a subject suffering from a disease or disorder comprising administering a compound of Formula (I) or a pharmaceutically acceptable salt thereof to the subject, wherein the subject is suffering from a disease or disorder selected from the group consisting of: cardiovascular disease including arteriosclerosis, peripheral vascular disease, thrombosis, ischemia-reperfusion events, congestive heart failure, primay and seconday pulmonary arterial hypertension and hypertension; renal diseases such as acute tubular necrosis; glomerulonephritis, including diabetic related complications including glomerular nephropathy and supportive care for dialysis including maintenance of arterial fistulas; pulmonary diseases including bronchitis, bronchiecstasis, chronic obstructive pulmonary disease, pulmonary edema, asthma, emphysema, sarcoidosis; liver disease including those leading to scarring and fibrosis such as cholestasis, hepatitis B and C infection, cirrhosis; autoimmune diseases and their complications including rheumatoid arthritis, ankylosing spondylosis, systemic lupus erthyamatosus, scleroderma and psoriasis; cerebral nerve degenerative diseases including Alzheimers disease, Parkinsons disease, ALS (amyotrophic lateral sclerosis) and multiple sclerosis; skin diseases; eye diseases including macular degeneration, cataracts, light retinopathy, and retinopathy of diabetes, prematurity; and cancer; supportive care for transplantation including graft viability and reduction of ischemic damage.

In each of the methods described above, a compound of Formula (I) or a pharmaceutically acceptable salt thereof may be administered to a subject as part of a pharmaceutically formulation as described above.

### EXAMPLES

The general procedures used in the methods to prepare the compounds of the present invention are described below.

### General Experimental Section

LC-MS data were obtained using gradient elution on a parallel MUX™ system, running four Waters® 1525 binary HPLC pumps, equipped with a Mux-UV 2488 multichannel UV-Vis detector (recording at 215 and 254 nM) and a Leap Technologies HTS PAL Auto sampler using a Sepax GP-C18, 4.6×50 mm; 5 micron particle-size column. A three minute gradient was run from 25% B (97.5% acetonitrile, 2.5% water, 0.05% TFA) and 75% A (97.5% water, 2.5% acetonitrile, 0.05% TFA) to 100% B. The system is interfaced with a Waters Micromass ZQ mass spectrometer using electrospray ionization. MassLynx software was employed. All MS data were obtained in the positive mode unless otherwise noted. ¹H NMR data were obtained on a Varian® Mercury 400 MHz spectrometer and chemical shifts were referenced using either the residual solvent proton signal (e.g., residual CHCl₃ in CDCl₃) or the TMS signal as an internal reference. Microwave heating procedures were used in some experiments and, in these cases, a Discover® microwave synthesis system (CEM, Matthews, N.C., USA) was used which included the use of pressurized glass reaction vessels at elevated temperatures.

All reagents and solvents including anhydrous solvents were commercially available and were used as received unless described otherwise. Any solutions of Grignard reagents and organolithium reagents were commercially available and were used as received and at the concentrations listed on their labels. Reactions are stirred using a magnetic stirring apparatus and magnetic stir bar in most cases. All reactions using air-sensitive reagents were run under inert gas. For reactions not heated using a microwave-generating apparatus, the reaction temperatures reported in the experimental section refer to the temperatures of an oil bath or cooling bath placed around a reaction vessel. For reactions performed using a microwave-generating apparatus, the temperatures refer to the temperatures reported by the microwave apparatus.

The compounds specifically exemplified below were named based on their chemical structure using Autonom 2000 (Version 4.1, SP1, Elsevier MDL) plug-in for ISIS Draw.

### Abbreviations

DCM=dichloromethane
DMAP±N,N′-dimethylamino pyridine
DME=1,2 dimethoxyethane
DMF=N,N′-dimethylformamide
DMSO=dimethylsulfoxide
EtOAc=ethyl acetate
LC/MS=liquid chromatography-mass spectrometry analysis
MeOH=methanol
THF=tetrahydrofuran
TLC=thin layer chromatography
rt or RT=room temperature
h=hour

### General Procedure A

The following is a stepwise general representative example for synthesis of 4-chloro quinolines 3-carboxylic acid esters.

Step 1: To a solution of o-anisidine (61.5 g, 500 mmol) in 200 mL of diphenylether was added diethyl ethoxymethylenemalonate (108 g, 1 eq., 500 mmol). The reaction mixture was heated to reflux and stirred at 90° C. for 4 h. The reaction byproduct ethanol was removed by distillation and the reaction mixture was then heated to reflux at 260° C. for 4 h. After completion of the reaction, the mixture was cooled down to rt, poured into water (300 mL) and extracted with DCM (2×500 mL). The combined organic extracts were washed with brine solution (2×250 mL) and concentrated under vacuum to give the crude product. The concentrated residue was then purified with silica gel chromatography using hexanes:ethyl acetate as an eluent (from 90:10 to 50:50) to afford 8-methoxy-4-oxo-1,4-dihydro-quinoline-3-carboxylic acid ethyl ester (111 g, 89.8%). LCMS: m/z 249 [M+2]. ¹H NMR (400 MHz, DMSO-d₆): δ 11.02 (d, 1H), 8.52 (d, 1H), 7.48 (d, 1H), 7.14 (t, 1H), 7.01 (m, 1H), 4.21 (q, 2H), 3.91 (s, 3H), and 1.25 (t, 3H).

Step 2: The product of step 1 (8-methoxy-4-oxo-1,4-dihydro-quinoline-3-carboxylic acid ethyl ester) (24.7 g, 100 mmol), was fully dissolved in phosphorus oxychloride (100 mL) and heated at 90° C. for 16 h. After completion of the reaction, the mixture was completely concentrated under reduced pressure and dissolved in DCM (250 mL). The organic mixture was then poured into cold saturated sodium bicarbonate solution (250 mL) and extracted with DCM. The organic extracts were washed with water (2×100 mL) and brine solution (2×100 mL), dried over sodium sulfate and concentrated under vacuum to give the desired product, 4-chloro-8-methoxy-quinoline-3-carboxylic acid ethyl ester (19.6 g, 73.9%). LCMS: m/z 267 [M+2]. ¹H NMR (400 MHz, CDCl₃): δ 9.18 (s, 1H), 7.96 (d, 1H), 7.60 (t, 1H), 7.18 (d, 1H), 4.50 (q, 2H), 4.12 (s, 3H), and 1.48 (t, 3H).

### General Procedure B:

The following is a general representative example for ipso substitution of 4-chloro quinolines.

To a solution of 4-chloro-8-methoxy-quinoline-3-carboxylic acid ethyl ester (265 mg, 1.0 mmol) in 20 mL of anhydrous THF was added cesium carbonate (750 mg, 2 eq., 2.0 mmol) and an alkylthiol (1.5 eq., 1.5 mmol) and heated at 70° C. for 16 h. After completion of the reaction, the mixture was cooled down to rt, poured into water (50 mL) and extracted with ethyl acetate (2×100 mL). The combined organic extracts were washed with water (2×50 mL), and brine solution (2×50 mL), and concentrated under vacuum to give the crude product. The concentrated residue was then purified with silica gel chromatography using hexanes:ethyl acetate as an eluent (from 80:20 to 50:50) to afford 4-alkylsulfanyl-8-methoxy-quinoline-3-carboxylic acid ethyl esters (65-92% yields). These products may then be treated to oxidation conditions to obtain 4-alkylsulfonyl-8-methoxy-quinoline-3-carboxylic acid ethyl esters.

### General Procedure C

The following is a general representative example for oxidation of 4-alklysulfanyl quinolines.

To a solution of an 4-alkylsulfonyl-8-methoxy-quinoline-3-carboxylic acid ethyl ester (0.5 mmol) in DCM (10 mL) was slowly added peracetic acid (2 eq., 32% wt in acetic acid, 1.0 mmol) at 0° C. and stirred for 2-4 h. The mixture was allowed to warm up to rt while stirring. After completion of the reaction, the mixture was poured into saturated sodium bicarbonate solution (50 mL), and extracted with DCM (2×50 mL). The organic extracts were combined, washed with water (2×50 mL), and brine solution (2×50 mL) and concentrated under vacuum to give the crude product. The concentrated residue was then purified with silica gel chromatography using hexanes:ethyl acetate as an eluent (from 80:20 to 50:50) to afford 4-alkylsulfonyl-8-methoxy-quinoline-3-carboxylic acid ethyl esters (45-78% yields).

### General Procedure D

The following is a general representative example for hydrolysis of 4-alklysulfanyl quinolines.

To a solution of 4-alkylsulfonyl-8-methoxy-quinoline-3-carboxylic acid ethyl ester (2.0 mmol) in THF:MeOH (1:1 v/v, 10 mL) was added lithium hydroxide (2.0 ml, 2 eq., 2.0 M solution in water, 4.0 mmol) at rt and stirred for 6 h. After completion of the reaction, the mixture was concentrated under vacuum then poured into 1.0 M HCl solution (20 mL) and extracted with ethyl acetate (2×50 mL). The organic extracts were combined, washed with water (2×50 mL), and brine solution (2×50 mL), dried over sodium sulfate and concentrated under vacuum to give the crude product (70-91% yields). These acids may then be subjected to further manipulations without purification.

### General Procedure E

The following is a general representative example for alkylation of 4-alklysulfanyl quinoline-3-carboxylic acids.

To a solution of 4-alkylsulfonyl-8-methoxy-quinoline-3-carboxylic acid (0.5 mmol) in either dioxane, THF or DMF (10 mL) were added cesium or potassium carbonate (2 eq., 1.0 mmol) and alkyl halide (1.5 eq., 1.5 mmol) and the reaction mixture was then heated to 80° C. and stirred for 4-12 h. The mixture was allowed to cool down to rt while stirring, poured into saturated sodium bicarbonate solution (50 mL), and extracted with ethyl acetate (2×50 mL). The organic extracts were combined, washed with water (2×50 mL) and brine solution (2×50 mL), dried over sodium sulfate, and concentrated under vacuum to give the crude product. The concentrated residue was then purified with silica gel chromatography using hexanes:ethyl acetate as an eluent (from 90:10 to 50:50) to afford 4-alkylsulfonyl-8-methoxy-quinoline-3-carboxylic acid alkyl esters (75-95% yields).

### General Procedure F

The following is a stepwise general representative example for formation of 3-iodo-8-methoxy-4-(2-methyl-tetrahydro-furan-3-ylsulfanyl)-quinoline.

Step 1. A 15% solution of iodine (8.27 mmol) in 20% aqueous potassium iodide (20 mL) was added dropwise to a stirred slurry of 4-hydroxyquinoline (1.0 g, 6.89 mmol) in 2N aqueous NaOH (15 mL). The reaction was stirred for 3 h at ambient temperature and was shown to be complete by TLC (1:1 hexanes-ethyl acetate). The mixture was then acidified with acetic acid, and the precipitate was filtered. It was then washed with water and dried under vacuum to yield 1.64 g of 3-iodo-quinolin-4-ol. This material was used in the next step without further purification. LCMS: m/z 273 (M+2)⁺ and ¹H NMR (DMSO-d₆, 400 MHz): δ 12.21 (br, 1H), 8.52 (s, 1H), 8.11 (d, 1H), 7.69 (t, 1H), 7.59 (d, 1H), 7.39 (t, 1H).

Step 2. To 3-iodo-quinolin-4-ol (500 mg, mol) in a 50 mL round bottom flask was added phosphorus oxychloride (2.5 mL). The reaction was heated at 90° C. for 2 h. LC/MS analysis showed a complete conversion to the product. After allowing the reaction to cool, ice-water was slowly added, and it was neutralized with dilute NaOH. The resulting precipitate was filtered and dried under vacuum to yield 450 mg of 4-chloro-3-iodo-quinoline. It was used in the next step without purification. LCMS: m/z 290 (M+1)⁺ and ¹H NMR (DMSO-d₆, 400 MHz): δ 9.19 (s, 1H), 8.25 (d, 1H), 8.10 (d, 1H), 7.91 (t, 1H), 7.79 (t, 1H).

Step 3. To a 1:1 THF-1,4-dioxane solution of 4-chloro-3-iodo-quinoline (459 mg, 1.59 mmol) was added 2-methyl-3-tetrahydrofuranthiol (225 mg, 1.90 mmol), followed by cesium carbonate (1.55 g, 4.77 mol). The reaction mixture was heated at 90° C. for 4 h, then at ambient temperature overnight. TLC analysis (4:1 hexanes-ethyl acetate) shows a complete consumption of the starting material. The reaction was diluted with EtOAc and water. The phases were separated, and the aqueous was extracted 2 times with EtOAc. The combined organics were dried over sodium sulfate and filtered, and the solvent was evaporated. The crude material was purified using silica gel chromatography eluting with 9:1 hexanes-EtOAc then 4:1 hexanes-EtOAc, yielding 300 mg of pure 3-iodo-4-(2-methyl-tetrahydro-furan-3-ylsulfanyl)-quinoline. An additional purification of the impure fractions yielded an additional 150 mg of pure material. LCMS: m/z 373 (M+2)⁺ and ¹H NMR (CDCl₃, 400 MHz): δ 9.23 (s, 1H), 8.62 (d, 1H), 8.11 (d, 1H), 7.78 (t, 1H), 7.64 (t, 1H), 3.97-3.90 (m, 3H), 3.48-3.39 (m, 1H), 2.31-2.19 (m, 1H), 2.02-1.94 (m, 1H), 1.11 (d, 3H).

### General Procedure G

The following is a general representative example for Suzuki coupling of 3-iodo-8-methoxy-4-(2-methyl-tetrahydro-furan-3-ylsulfanyl)-quinoline with various arylboronic acids.

To a DME solution of 3-iodo-4-(2-methyl-tetrahydro-furan-3-ylsulfanyl)-quinoline (100 mg, 0.269 mmol) was added (4-methoxyphenyl)boronic acid (61 mg, 0.404 mmol), followed by 2N sodium carbonate (269 uL, 0.538 mmol) and Pd(PPh₃)₄ (23 mg, 0.02 mmol). The reaction was heated at 90° C. for 16 h. LC/MS analysis showed a complete consumption of the starting material. It was allowed to return to ambient temperature and filtered, and the solvent was evaporated. The crude material was purified using silica gel chromatography, yielding 100 mg of pure 3-(4-methoxy-phenyl)-4-(2-methyl-tetrahydro-furan-3-ylsulfanyl)-quinoline.

LC/MS: m/z 353 (M+2)⁺.

### Example 1

To a solution of 4-chloro-8-methoxy-quinoline-3-carboxylic acid ethyl ester (265 mg, 1.0 mmol), which was synthesized using a procedure analogous to general procedure A, in 20 mL of DMF was added catalytic amount of DMAP (11 mg, 10% mol, 0.1 mmol) and methanasulfinic acid sodium salt (204 mg, 2 eq., 2.0 mmol) and heated at 120° C. for 16 h. After completion of the reaction, the mixture was cooled down to rt and poured into water (50 mL) and extracted with ethyl acetate (2×100 mL) and the organic extracts were combined, washed with water (2×50 mL), and brine solution (2×50 mL), concentrated under vacuum to give the crude product. The concentrated residue was then purified with silica gel chromatography using hexanes:ethyl acetate as an eluent system (from 80:20 to 50:50) to afford 4-methanesulfonyl-8-methoxy-quinoline-3-carboxylic acid ethyl ester (143 mg). LCMS: m/z 311 [M+2].

### Example 2

8-Methoxy-4-(2-methyl-tetrahydro-furan-3-sulfonyl)-quinoline-3-carboxylic acid ethyl ester (213 mg) was prepared from 4-chloro-8-methoxy-quinoline-3-carboxylic acid ethyl ester (265 mg, 1.0 mmol) and 2-methyl-3-tetrahydrofuranthiol (240 mg, 2.0 eq, 2.0 mmol) using a procedure analogous to general procedures B and C. LCMS: m/z 381 [M+2]. ¹H NMR (400 MHz, CDCl₃): δ 9.58 (s, 1H), 8.12 (d, 1H), 7.28 (t, 1H), 7.10 (d, 1H), 4.48 (q, 2H), 4.02 (s, 3H), 3.82 (q, 1H), 2.26 (m, 3H), 1.84 (m, 4H), 1.40 (t, 3H) and 1.24 (m, 1H).

### Example 3

4-Cyclohexanesulfonyl-8-methoxy-quinoline-3-carboxylic acid ethyl ester (196 mg) was prepared from 4-chloro-8-methoxy-quinoline-3-carboxylic acid ethyl ester (265 mg, 1.0 mmol) and cyclohexanethiol (232 mg, 2.0 eq, 2.0 mmol) using a procedure analogous to general procedures B and C. LCMS: m/z 379 [M+2].

### Example 4

8-Methoxy-4-(2-methyl-furan-3-sulfonyl)-quinoline-3-carboxylic acid ethyl ester (173 mg) was prepared from 4-chloro-8-methoxy-quinoline-3-carboxylic acid ethyl ester (265 mg, 1.0 mmol) and 2-methyl-3-furanthiol (228 mg, 2.0 eq, 2.0 mmol) using a procedure analogous to general procedures B and C. LCMS: m/z 377 [M+2]. ¹H NMR (400 MHz, CDCl₃): δ 8.09 (d, 1H), 7.98 (d, 1H), 7.58 (dd, 1H), 7.41 (t, 1H), 7.23 (d, 1H), 6.78 (d, 1H), 4.54 (q, 2H), 4.01 (s, 3H), 2.67 (s, 3H), and 1.46 (t, 3H).

### Example 5

4-(Furan-3-ylmethanesulfonyl)-8-methoxy-quinoline-3-carboxylic acid ethyl ester (218 mg) was prepared from 4-chloro-8-methoxy-quinoline-3-carboxylic acid ethyl ester (265 mg, 1.0 mmol) and furan-3-yl-methanethiol (228 mg, 2.0 eq, 2.0 mmol) using a procedure analogous to general procedures B and C. LCMS: m/z 376 [M+1].

### Example 6

4-(2-Methyl-tetrahydro-furan-3-sulfonyl)-quinoline-3-carboxylic acid ethyl ester (196 mg) was prepared from 4-chloroquinoline-3-carboxylic acid ethyl ester (235 mg, 1.0 mmol) and 2-methyl-3-tetrahydrofuranthiol (235 mg, 2.0 eq, 2.0 mmol) using a procedure analogous to general procedures B and C. LCMS: m/z 350 [M+1].

### Example 7

The starting material, 1-(4-chloro-8-methoxy-quinolin-3-yl)-butan-1-one (1.87 g) was prepared from o-anisidine (1.23 g, 10.0 mmol) and 2-[1-ethoxy-meth-(Z)-ylidene]-3-oxo-hexanoic acid ethyl ester (2.1 g, 10.0 mmol) following the general procedure A. Then 1-[8-methoxy-4-(2-methyl-tetrahydro-furan-3-sulfonyl)-quinolin-3-yl]-butan-1-one (243 mg, 64%) was prepared from 1-(4-chloro-8-methoxy-quinolin-3-yl)-butan-1-one (263 mg, 1.0 mmol) and 2-methyl-3-tetrahydrofuranthiol (228 mg, 2.0 eq, 2.0 mmol) using a procedure analogous to general procedures B and C. LCMS: m/z 378 [M+1].

### Example 8

8-Methoxy-4-(2-methyl-tetrahydro-furan-3-sulfonyl)-quinoline-3-carboxylic acid cyclohexyl ester (196 mg) was prepared from 8-methoxy-4-(2-methyl-tetrahydro-furan-3-ylsulfanyl)-quinoline-3-carboxylic acid (160 mg, 0.5 mmol) and cyclohexyl bromide (120 mg, 1.5 eq., 0.75 mmol) using a procedure analogous to the general procedures E and C. LCMS: m/z 435 [M+2].

### Example 9

8-Methoxy-4-(2-methyl-tetrahydro-furan-3-sulfonyl)-quinoline-3-carboxylic acid cyclohexylmethyl ester (229 mg) was prepared from 8-methoxy-4-(2-methyl-tetrahydro-furan-3-ylsulfanyl)-quinoline-3-carboxylic acid (160 mg, 0.5 mmol) and cyclohexylmethyl bromide (132 mg, 1.5 eq., 0.75 mmol) using a procedure analogous to the general procedures E and C. LCMS: m/z 449 [M+2].

### Example 10

8-Methoxy-4-(2-methyl-tetrahydro-furan-3-sulfonyl)-quinoline-3-carboxylic acid 3-methoxy-propyl ester (183 mg) was prepared from 8-methoxy-4-(2-methyl-tetrahydro-furan-3-ylsulfanyl)-quinoline-3-carboxylic acid (160 mg, 0.5 mmol) and 3-methoxypropyl bromide (115 mg, 1.5 eq., 0.75 mmol) using a procedure analogous to the general procedures E and C.

LCMS: m/z 425 [M+2]. ¹H NMR (400 MHz, CDCl₃): δ 8.96 (s, 1H), 8.48 (d, 1H), 7.74 (t, 1H), 7.23 (d, 1H), 4.58 (t, 2H), 4.48 (t, 1H), 4.18 (m, 2H), 4.14 (s, 3H), 3.68 (q, 1H), 3.52 (t, 2H), 3.36 (s, 3H), 2.51 (m, 1H), 2.08 (p, 2H), 1.85 (m, 1H), and 1.72 (d, 3H).

### Example 11

8-Methoxy-4-(2-methyl-tetrahydro-furan-3-sulfonyl)-quinoline-3-carboxylic acid tetrahydro-furan-2-ylmethyl ester (205 mg) was prepared from 8-methoxy-4-(2-methyl-tetrahydro-furan-3-ylsulfanyl)-quinoline-3-carboxylic acid (160 mg, 0.5 mmol) and tetrahydrofurfuryl bromide (124 mg, 1.5 eq., 0.75 mmol) using a procedure analogous to general procedures E and C. LCMS: m/z 437 [M+2].

### Example 12

8-Methoxy-4-(2-methyl-furan-3-sulfonyl)-quinoline-3-carboxylic acid 3-methoxy-propyl ester (210 mg) was prepared from 8-methoxy-4-(2-methyl-furan-3-ylsulfanyl)-quinoline-3-carboxylic acid (157 mg, 0.5 mmol) and 3-methoxypropyl bromide (115 mg, 1.5 eq., 0.75 mmol) using a procedure analogous to general procedures E and C. LCMS: m/z 420 [M+1].

### Example 13

4-(Furan-3-ylmethanesulfonyl)-8-methoxy-quinoline-3-carboxylic acid 3-methoxy-propyl ester (191 mg) was prepared from 4-(furan-3-ylmethanesulfonyl)-8-methoxy-quinoline-3-carboxylic acid (157 mg, 0.5 mmol) and 3-methoxypropyl bromide (115 mg, 1.5 eq., 0.75 mmol) using a procedure analogous to general procedures E and C. LCMS: m/z 420 [M+1].

### Example 14

4-(2-Methyl-tetrahydro-furan-3-sulfonyl)-quinoline-3-carboxylic acid 3-methoxy-propyl ester (204 mg) was prepared from 4-(2-methyl-tetrahydro-furan-3-ylsulfanyl)-quinoline-3-carboxylic acid (145 mg, 0.5 mmol) and 3-methoxypropyl bromide (115 mg, 1.5 eq., 0.75 mmol) using a procedure analogous to general procedures E and C. LCMS: m/z 394 [M+1].

### Example 15

8-Methoxy-4-(2-methyl-furan-3-sulfonyl)-quinoline-3-carboxylic acid tetrahydro-furan-2-ylmethyl ester (211 mg) was prepared from 8-methoxy-4-(2-methyl-furan-3-ylsulfanyl)-quinoline-3-carboxylic acid (158 mg, 0.5 mmol) and tetrahydrofurfuryl bromide (124 mg, 1.5 eq., 0.75 mmol) using a procedure analogous to general procedures E and C. LCMS: m/z 432 [M+1].

### Example 16

4-(Furan-3-ylmethanesulfonyl)-8-methoxy-quinoline-3-carboxylic acid tetrahydro-furan-2-ylmethyl ester (215 mg) was prepared from 4-(furan-3-ylmethanesulfonyl)-8-methoxy-quinoline-3-carboxylic acid (158 mg, 0.5 mmol) and tetrahydrofurfuryl bromide (124 mg, 1.5 eq., 0.75 mmol) using a procedure analogous to general procedures E and C. LCMS: m/z 432 [M+1].

### Example 17

4-(2-Methyl-tetrahydro-furan-3-sulfonyl)-quinoline-3-carboxylic acid tetrahydro-furan-2-ylmethyl ester (174 mg) was prepared from 4-(2-methyl-tetrahydro-furan-3-ylsulfanyl)-quinoline-3-carboxylic acid (145 mg, 0.5 mmol) and tetrahydrofurfuryl bromide (124 mg, 1.5 eq., 0.75 mmol) using a procedure analogous to general procedures E and C. LCMS: m/z 406 [M+2].

### Example 18

8-Methoxy-4-(2-methyl-furan-3-sulfonyl)-quinoline-3-carboxylic acid pentyl ester (197 mg) was prepared from 8-methoxy-4-(2-methyl-furan-3-ylsulfanyl)-quinoline-3-carboxylic acid (157 mg, 0.5 mmol) and pentyl bromide (112 mg, 1.5 eq., 0.75 mmol) using a procedure analogous to general procedures E and C. LCMS: m/z 419 [M+2].

### Example 19

8-Methoxy-4-(2-methyl-furan-3-sulfonyl)-quinoline-3-carboxylic acid cyclopentylmethyl ester (202 mg) was prepared from 8-methoxy-4-(2-methyl-furan-3-ylsulfanyl)-quinoline-3-carboxylic acid (158 mg, 0.5 mmol) and cyclopentylmethyl bromide (120 mg, 1.5 eq., 0.75 mmol) using a procedure analogous to general procedures E and C. LCMS: m/z 431 [M+2].

### Example 20

3-Iodo-4-(2-methyl-tetrahydro-furan-3-ylsulfanyl)-quinoline (283 mg) was prepared from 4-chloro-3-iodo-quinoline (290 mg, 1.0 mmol) following the general procedure F. This crude product (75 mg, 0.2 mmol) was then subjected to alklylation while heating with 2-methyl-3-tetrahydrofuranthiol (60 mg, 0.5 mmol) and cesium carbonate (160 mg, 0.5 mmol) in DMF (10 mL) to give bis-alkylated product, 3,4-bis-(2-methyl-tetrahydro-furan-3-ylsulfanyl)-quinoline (58 mg).

Then, 4-(2-methyl-tetrahydro-furan-3-sulfonyl)-3-(2-methyl-tetrahydro-furan-3-ylsulfanyl)-quinoline (30 mg) was prepared from 3,4-bis-(2-methyl-tetrahydro-furan-3-ylsulfanyl)-quinoline (58 mg, 0.16 mmol) using a procedure analogous to the general procedure C. LCMS: m/z 395 [M+2].

### Example 21

4-(2-Methyl-tetrahydro-furan-3-sulfonyl)-nicotinic acid 3-methoxy-propyl ester (98 mg) was prepared from 4-(2-methyl-tetrahydro-furan-3-sulfonyl)nicotinic acid (120 mg, 0.5 mmol) and 3-methoxypropyl bromide (115 mg, 1.5 eq., 0.75 mmol) using a procedure analogous to the general procedures E and C. LCMS: m/z 344 [M+1].

### Example 22

4-(2-Methyl-tetrahydro-furan-3-sulfonyl)-1-oxy-nicotinic acid 3-methoxy-propyl ester (29 mg) was prepared from 4-(2-methyl-tetrahydro-furan-3-sulfonyl)nicotinic acid (60 mg, 0.25 mmol) and 3-methoxypropyl bromide (58 mg, 1.5 eq., 0.38 mmol) using a procedure analogous to general procedures E and C. LCMS: m/z 360 [M+1].

### Example 23

4-(Propane-1-sulfonyl)-nicotinic acid 3-methoxy-propyl ester (73 mg) was prepared from 4-(propane-1-sulfonyl)-nicotinic acid (98 mg, 0.5 mmol) and 3-methoxypropyl bromide (115 mg, 1.5 eq., 0.75 mmol) using a procedure analogous to the general procedures E and C. LCMS: m/z 303 [M+2].

### Example 24

4-Cyclohexylmethanesulfonyl-nicotinic acid 3-methoxy-propyl ester (73 mg) was prepared from 4-cyclohexylmethanesulfonyl-nicotinic acid (126 mg, 0.5 mmol) and 3-methoxypropyl bromide (115 mg, 1.5 eq., 0.75 mmol) using a procedure analogous to the general procedures E and C. LCMS: m/z 357 [M+2].

### Example 25

3-Iodo-4-(2-methyl-tetrahydro-furan-3-ylsulfanyl)-quinoline (283 mg) was prepared from 4-chloro-3-iodo-quinoline (290 mg, 1.0 mmol) using a procedure analogous to the general procedure F. After that this material (186 mg, 0.5 mmol) was dissolved in anhydrous THF (5 mL), to this solution were added [Pd(PPh₃) Cl₂] (9 mg, 10% mol, 0.05 mmol), copper iodide (5 mg, 10% mol, 0.05 mmol), triethylamine (0.1 mL, 1.5 eq., 0.75 mmol), and 3-methoxy propyne (42 mg, 1.2 eq., 0.6 mmol).

The reaction mixture stirred at rt for 16 h. Upon completion of the reaction, the mixture was poured into diluted HCl solution (10 mL, 1.0 N solution in water) and extracted with ethyl acetate (2×25 mL). The organic extracts were combined, washed with water (2×25 mL) and brine (2×25 mL) and concentrated under vacuum. This residue was then purified with silica gel chromatography using hexanes:ethyl acetate (from 95:5 to 80:20) as eluent system to give 3-(3-methoxy-prop-1-ynyl)-4-(2-methyl-tetrahydro-furan-3-sulfanyl)-quinoline (118 mg, 76%). Then, 3-(3-methoxy-prop-1-ynyl)-4-(2-methyl-tetrahydro-furan-3-sulfonyl)-quinoline (57 mg) was prepared from 3-(3-methoxy-prop-1-ynyl)-4-(2-methyl-tetrahydro-furan-3-sulfanyl)-quinoline (78 mg, 0.25 mmol) following the general procedure C. LCMS: m/z 347 [M+2].

### Example 26

3-Iodo-4-(2-methyl-tetrahydro-furan-3-ylsulfanyl)-quinoline (283 mg) was prepared from 4-chloro-3-iodo-quinoline (290 mg, 1.0 mmol) using a procedure analogous to the general procedure F. After that this material (186 mg, 0.5 mmol) was dissolved in anhydrous DMF (10 mL), to this solution were added palladium acetate (5 mg, 5% mol, 0.05 mmol), potassium acetate (42 mg, 0.5 mmol), tetrabutylammonium chloride (75 mg, 0.5 eq., 0.25 mmol), and ethyl acrylate (50 mg, 0.5 mmol).

The reaction mixture was heated to 110° C. and stirred at this temperature for 16 h. Upon completion of the reaction, the mixture was poured into saturated ammonium chloride solution (25 mL) and extracted with ethyl acetate (2×25 mL). The organic extracts were combined, washed with water (2×25 mL) and brine (2×25 mL), dried over sodium sulfate and concentrated under vacuum. This residue was then purified with silica gel chromatography using hexanes:ethyl acetate (from 95:5 to 70:30) as eluent system to give (E)-3-[4-(2-methyl-tetrahydro-furan-3-sulfanyl)-quinolin-3-yl]acrylic acid ethyl ester (98 mg, 57%). Then, (E)-3-[4-(2-methyl-tetrahydro-furan-3-sulfonyl)-quinolin-3-yl]acrylic acid ethyl ester (49 mg) was prepared from (E)-3-[4-(2-methyl-tetrahydro-furan-3-sulfanyl)-quinolin-3-yl]-acrylic acid ethyl ester (86 mg, 0.25 mmol) following the general procedure C. LCMS: m/z 377 [M+2].

### Example 27

3-Iodo-4-(2-methyl-tetrahydro-furan-3-ylsulfanyl)-quinoline (283 mg) was prepared from 4-chloro-3-iodo-quinoline (290 mg, 1.0 mmol) using a procedure analogous to the general procedure F. Then, 3-(4-trifluoromethyl-phenyl)-4-(2-methyl-tetrahydro-furan-3-sulfonyl)-quinoline (56 mg) was prepared from 3-iodo-4-(2-methyl-tetrahydro-furan-3-ylsulfanyl)-quinoline (100 mg, 0.27 mmol) and (4-trifluoromethyl-phenyl)boronic acid (80 mg, 0.4 mmol) using a procedure analogous to general procedures G and C. LC/MS: m/z 423 [M+2].

### Example 28

3-Iodo-4-(2-methyl-tetrahydro-furan-3-ylsulfanyl)-quinoline (283 mg) was prepared from 4-chloro-3-iodo-quinoline (290 mg, 1.0 mmol) using a procedure analogous to the general procedure F. Then, 3-(4-methoxy-phenyl)-4-(2-methyl-tetrahydro-furan-3-sulfonyl)-quinoline (56 mg) was prepared from 3-iodo-4-(2-methyl-tetrahydro-furan-3-ylsulfanyl)-quinoline (100 mg, 0.27 mmol) and (4-methoxyphenyl)boronic acid (61 mg, 0.4 mmol) using a procedure analogous to general procedures G and C. LC/MS: m/z 385 [M+2]. ¹H NMR (CDCl₃, 400 MHz): δ 9.43-9.27 (m, 1H), 8.82 (t, 1H), 8.44 (d, 1H), 7.89-7.72 (m, 2H) 7.47-7.36 (m, 2H) 7.06-6.99 (m, 2H), 4.41-4.29 (br, 1H), 3.90 (s, 3H), 3.87-3.80 (m, 1H), 3.79-3.65 (m, 2H), 2.98-2.87 (m, 1H), 2.24-2.04 (m, 1H), 0.90 (d, 3H).

### Example 29

Step 1: To methyl bromoacetate (1.0 mL, 10.56 mmol) in methanol (30 mL) was added sodium thiopropoxide (1.05 g, 10.56 mmol), and the mixture was stirred at room temperature for 12 h. The reaction mixture was concentrated, followed by the addition of water, and extraction with ethyl acetate. The organic layer was dried over sodium sulfate and concentrated to provide propylsulfanyl-acetic acid methyl ester.

Step 2: Propylsulfanyl-acetic acid methyl ester (1.3 g, 8.78 mmol) in diethyl ether (10 mL) was added dropwise to an ice-cold, stirred suspension of sodium methoxide (0.475 g, 8.78 mmol) in dry diethyl ether (10 mL). The mixture was stirred at 0° C. for 1 h and then treated dropwise with a solution of methyl formate (0.6 mL, 9.66 mmol). The mixture was stirred at 0° C. for 1 h and then at room temperature overnight. Water (30 mL) was added, the mixture was equilibrated, and the two layers were separated. The aqueous layer was added to a cold solution (0° C.) of 4-methoxy-phenylamine (1.08 g, 8.78 mmol) in water (20 mL) containing 10 N HCl (5 mL). The mixture was stirred for 1 h at 0° C. and then allowed to reach room temperature and extracted with dichloromethane. The dichloromethane extracts were washed with water, dried on sodium sulfate, and evaporated to get 3-(4-methoxy-phenylamino)-2-propylsulfanyl-acrylic acid methyl ester as oil. Thus obtained 3-(4-methoxy-phenylamino)-2-propylsulfanyl-acrylic acid methyl ester was dissolved in diphenylether (6 mL) and microwaved at 200° C. for 80 min. The reaction mixture was loaded on to silica gel column and eluted with methanol in dichloromethane (0% to 10% methanol gradient) to get 6-methoxy-3-propylsulfanyl-quinolin-4-ol. Yield: 300 mg.

Step 3: 6-Methoxy-3-propylsulfanyl-quinolin-4-ol (300 mg, 1.2 mmol) was dissolved in phosphorous oxychloride (5 mL) and stirred at room temperature for 3 h. The reaction mass was divided into two parts and concentrated. One part was added cyclopentylamine and microwaved at 150° C. for 60 min. Reaction mixture was loaded on to silica gel column and eluted with ethyl acetate (10-30% gradient) in hexanes to provide cyclopentyl-(6-methoxy-3-propylsulfanyl-quinolin-4-yl)-amine (yield: 50 mg).

Step 4: To the cyclopentyl-(6-methoxy-3-propylsulfanyl-quinolin-4-yl)-amine (50 mg, 0.158 mmol) in dichloromethane (2 mL) was added peracetic acid in acetic acid (32%, 0.19 mmol) and stirred for 30 min at room temperature. The reaction mixture was loaded on to silica gel column and eluted with ethyl acetate (20% to 100%) in hexanes to provide cyclopentyl-[6-methoxy-3-(propane-1-sulfonyl)-quinolin-4-yl]amine. Yield: 25 mg. LCMS: m/z 350.0 [M+2]. ¹H NMR (400 MHz, CDCl₃): δ 1.0 (t, 3H), 1.6-2.2 (m, 10H), 3.1 (m, 2H), 3.92 (s, 3H), 4.55 (m, 1H), 7.41 (dd, 1H), 7.44 (br, 1H), 7.56 (d, 1H), 7.92 (d, 1H), 8.72 (s, 1H).

### Example 30

Step 1: To the crude 4-chloro-6-methoxy-3-propylsulfanyl-quinoline obtained in Example 29 was added cyclohexylamine, and the mixture was microwaved at 150° C. for 60 min. The reaction mixture was loaded on to silica gel column and eluted with ethyl acetate (10-30% gradient) in hexanes to provide cyclohexyl-(6-methoxy-3-propylsulfanyl-quinolin-4-yl)-amine (yield: 25 mg).

Step 2: To the cyclohexyl-(6-methoxy-3-propylsulfanyl-quinolin-4-yl)-amine (25 mg, 0.076 mmol) in dichloromethane (2 mL) was added peracetic acid in acetic acid (32%, 0.09 mmol), and the mixture was stirred for 30 min at room temperature. The reaction mixture was loaded on to silica gel column and eluted with ethyl acetate (20% to 100%) in hexanes to get cyclohexyl-[6-methoxy-3-(propane-1-sulfonyl)-quinolin-4-yl]-amine (yield: 15 mg). LCMS: m/z 364.0 [M+2].

### Example 31

Step 1: To methyl bromoacetate (1.0 mL, 10.56 mmol) in methanol (30 mL) added sodium thiopropoxide (1.05 g, 10.56 mmol) and stirred at room temperature for 12 h. Concentrated the reaction mass, added water and extracted into ethyl acetate.

Dried the organic layer on sodium sulfate and concentrated to get propylsulfanyl-acetic acid methyl ester.

Step 2: Propylsulfanyl-acetic acid methyl ester (1.3 g, 8.78 mmol) in diethyl ether (10 mL) was added dropwise to an ice-cold, stirred suspension of sodium methoxide (0.475 g, 8.78 mmol) in dry diethyl ether (10 mL). The mixture was stirred at 0° C. for 1 h and then treated dropwise with a solution of methyl formate (0.6 mL, 9.66 mmol). The mixture was stirred at 0° C. for 1 h and then at room temperature overnight. Water (30 mL) was added, the mixture was equilibrated, and the two layers were separated. The aqueous layer was added to a cold solution (0° C.) of 2-trifluoromethoxy-phenylamine (1.55 g, 8.78 mmol) in water (20 mL) containing 10 N HCl (5 mL). The mixture was stirred for 1 h at 0° C. and then allowed to reach room temperature and extracted with dichloromethane. The dichloromethane extracts were washed with water, dried on sodium sulfate, and evaporated to get 2-propylsulfanyl-3-(2-trifluoromethoxy-phenylamino)-acrylic acid methyl ester as oil. Thus obtained 2-propylsulfanyl-3-(2-trifluoromethoxy-phenylamino)-acrylic acid methyl ester was dissolved in diphenylether (6 mL) and microwaved at 200° C. for 80 min. Loaded the reaction mixture on to silica gel column and eluted with methanol in dichloromethane (0% to 10% methanol gradient) to provide 3-propylsulfanyl-8-trifluoromethoxy-quinolin-4-ol. Yield: 400 mg.

Step 3: 3-Propylsulfanyl-8-trifluoromethoxy-quinolin-4-ol (400 mg, 1.32 mmol) was dissolved in phosphorous oxychloride (5 mL) and stirred at room temperature for 3 h. The reaction mass was divided into two parts and concentrated. To one part was added cyclopentylamine, and the mixture was microwaved at 150° C. for 60 min. The reaction mixture was loaded on to silica gel column and eluted with ethyl acetate (10-20% gradient) in hexanes to provide cyclopentyl-(3-propylsulfanyl-8-trifluoromethoxy-quinolin-4-yl)-amine (yield: 150 mg).

Step 4: To the cyclopentyl-(3-propylsulfanyl-8-trifluoromethoxy-quinolin-4-yl)-amine (150 mg, 0.405 mmol) in dichloromethane (2 mL) was added peracetic acid in acetic acid (32%, 0.0.486 mmol) and stirred for 30 min at room temperature. The reaction mixture was loaded on to silica gel column and eluted with ethyl acetate (10% to 33%) in hexanes to provide cyclopentyl-[3-(propane-1-sulfonyl)-8-trifluoromethoxy-quinolin-4-yl]-amine (yield: 60 mg). LCMS: m/z 403.9 [M+2]. ¹H NMR (400 MHz, CDCl₃): δ 1.0 (t, 3H), 1.6-1.9 (m, 8H), 2.05-2.4 (m, 2H), 3.1 (m, 2H), 4.5 (m, 1H), 7.43 (dd, 1H), 7.64 (m, 1H), 7.82 (br, 1H), 8.21 (dd, 1H), 8.9 (s, 1H).

### Example 32

Step 1: To the crude 4-chloro-3-propylsulfanyl-8-trifluoromethoxy-quinoline obtained in step-3 of example 31 was added cyclohexylamine, and the mixture was microwaved at 150° C. for 60 min. The reaction mixture was loaded on to silica gel column and eluted with ethyl acetate (10-20% gradient) in hexanes to provide cyclohexyl-(3-propylsulfanyl-8-trifluoromethoxy-quinolin-4-yl)-amine (yield: 175 mg).

Step 2: To the cyclohexyl-(3-propylsulfanyl-8-trifluoromethoxy-quinolin-4-yl)-amine (175 mg, 0.46 mmol) in dichloromethane (2 mL) was added peracetic acid in acetic acid (32%, 0.55 mmol), and the mixture was stirred for 30 min at room temperature. The reaction mixture was loaded on to a silica gel column and eluted with ethyl acetate (10% to 33%) in hexanes to provide cyclohexyl-[3-(propane-1-sulfonyl)-8-trifluoromethoxy-quinolin-4-yl]-amine (yield: 65 mg). LCMS: m/z 418.0 [M+2]. ¹H NMR (400 MHz, CDCl₃): δ 1.02 (t, 3H), 1.4-1.55 (m, 4H), 1.6-1.9 (m, 6H), 2.05-2.3 (m, 2H), 3.12 (m, 2H), 3.98 (m, 1H), 7.45 (dd, 1H), 7.55 (brd, 1H), 7.66 (m, 1H), 8.21 (dd, 1H), 8.91 (s, 1H).

### Biological Assay

Oxidative Stress Gene Expression (mRNA) Protocol

Cell Culture. Normal Human Fibroblast (NHLF), were obtained from Lonza. Cultures of cells were maintained in Fibroblast Growth Medium (FGM-2) medium supplemented with 2% fetal bovine serum (FBS), Fibroblast Growth Factor (hFGF-B) 0.5 ml, Insulin 0.5 ml, gentamicin/amphotericin-B 0.5 mL at 37° C. in a humidified atmosphere of 5% CO₂. Primary isolates of cell are cultured in T-225 flasks (Corning) as per instructions from the vendor. Cells are grown to 80% confluence before harvesting. Cells are harvested by first washing with 5 mL of Hepes buffered saline solution (Lonza). The buffered salt solution is aspirated and then 5 mL of a solution of Trypsin-EDTA is added to detach the cells from the culture flask. To this mixture 5 mL of Trypsin neutralization solution (Lonza) is added Trypsin. The mixture is centrifuged at 500×g for 10 minutes at 4° C. The cell pellet is resuspended to a final concentration of one million cells per milliliter in fetal bovine serum containing 6% DMSO. The cells are then dispensed into cyrovials and frozen in liquid nitrogen for storage.

Heme oxygenase mRNA expression. Secondary growth of NHLF cells are used for all assays. Cell are thawed and then placed into culture in T225 flasks. The cells are grown to 80% confluency and harvested by washing once with HEPES buffered saline solution and then trypsinized. Equal numbers (7×10³ cells per well) of NHLF cells were plated in 96 well tissue culture plates (Corning). Cells were incubated overnight in FGM-2 medium prior to exposure to compound. Cells were treated with either DMSO or a compound of the Examples above for 6 h. Preparation of cell lysates was carried out according to instructions using the Quantigene Reagent system 2.0 protocol (Panomics). Heme oxygenase mRNA induction was detected from cell lysate using the Quantigene Plex 2.0 Assay Kit (Panomics) on the Luminex platform (BIO-RAD). Expression of GAPDH mRNA was used as the internal control for the Quantigen Plex 2.0 Assay. The data in Table 1 represents the fold change of heme oxygenase mRNA expression, normalized to control values, above DMSO treated cells.

We claim:

1. A compound of Formula (I) or a pharmaceutically acceptable salt thereof: 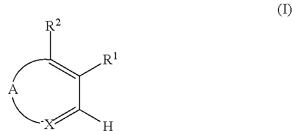
wherein
A is 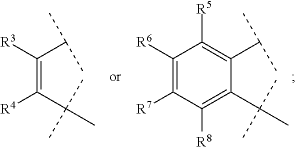
X is N or N—O;
R¹ is the group -D¹-L¹-R¹¹, wherein
D¹ is selected from the group consisting of: direct bond, —C(O)—, —CO₂—, —NH—C(O)—, —S—, —S(O)—, —SO₂—, —CH═CH—CO₂—, and —C≡C—CH₂—,
L¹ is selected from the group consisting of: direct bond and C₁₋₆ alkylene, and
R¹¹ is selected from the group consisting of: hydrogen, C₁₋₆ alkyl, cyclopentyl, cyclohexyl, —O—C₁₋₆ alkyl, tetrahydrofuran-2-yl, tetrahydrofuran-3-yl, and phenyl, wherein the cyclopentyl, cyclohexyl, tetrahydrofuanyl, and phenyl groups are optionally substituted with one or more substituents independently selected from the group consisting of: C₁₋₆ alkyl, C₁₋₆ haloalkyl, —O—C₁₋₆alkyl, —O—C₁₋₆ haloalkyl, and halogen,
wherein, when D¹ and L¹ are direct bonds, then R¹¹ is a phenyl group;
R² is the group -D²-L²-R¹², wherein
D² is selected from the group consisting of: —S—, —SO₂—, and —NH—,
L² is selected from the group consisting of: direct bond and C₁₋₆ alkylene, and
R¹¹ is selected from the group consisting of: C₁₋₆ alkyl, cyclopentyl, cyclohexyl, —O—C₁₋₆ alkyl, furan-2-yl, furan-3-yl, tetrahydrofuran-2-yl, tetrahydrofuran-3-yl, pyridine-2-yl, and phenyl, wherein the cyclopentyl, cyclohexyl, furanyl, tetrahydrofuanyl, pyridine, and phenyl groups are optionally substituted with one or more substituents independently selected from the group consisting of: C₁₋₆ alkyl, C₁₋₆ haloalkyl, —O—C₁₋₆alkyl, —O—C₁₋₆ haloalkyl, and halogen;
R³, R⁴, R⁵, R⁶, R⁷, and R⁸ are independently selected from the group consisting of hydrogen, —O—C₁₋₆ alkyl, and —O—C₁₋₆ haloalkyl.

2. The compound according to claim 1,
wherein A is 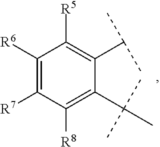
and X is N.

3. The compound according to claim 2, wherein R⁵ and R⁷ are hydrogen, and R⁶ and R⁸ are independently selected from the group consisting of: hydrogen, —O—C₁₋₆ alkyl, and —O—C₁₋₆ haloalkyl.

4. The compound according to claim 3, wherein R¹ is the group -D¹-L¹-R¹¹, wherein
D¹ is selected from the group consisting of: direct bond, —C(O)—, —CO₂—, —NHC(O)—, —S—, —S(O)—, —SO₂—, —CH═CH—CO₂—, and —C≡C—CH₂—,
L¹ is selected from the group consisting of: direct bond and C₁₋₆ alkylene, and
R¹¹ is selected from the group consisting of: C₁₋₆ alkyl, cyclopentyl, cyclohexyl, —O—C₁₋₆ alkyl, tetrahydrofuran-2-yl, tetrahydrofuran-3-yl, and phenyl, wherein the cyclopentyl, cyclohexyl, tetrahydrofuanyl, and phenyl groups are optionally substituted with one or more substituents independently selected from the group consisting of: C₁₋₆ alkyl, C₁₋₆ haloalkyl, —O—C₁₋₆alkyl, —O—C₁₋₆ haloalkyl, and halogen.

5. The compound according to claim 1, wherein D¹ is —CO₂—, L¹ is a C₁₋₆ alkylene group.

6. The compound according to claim 5, wherein R¹¹ is a —O—C₁₋₆ alkyl group.

7. The compound according to claim 5, wherein R² is the group -D²-L²-R¹², wherein D² is —SO₂—.

8. The compound according to claim 5, wherein L² is a direct bond or a C₁₋₃ alkylene group, and R¹² is selected from the group consisting of: furan-2-yl, furan-3-yl, tetrahydrofuran-2-yl, and tetrahydrofuran-3-yl, wherein the furanyl and tetrahydrofuanyl groups are optionally substituted with one or more substituents independently selected from the group consisting of: C₁₋₆ alkyl.

9. The pharmaceutical composition comprising a compound according to claim 1 and a pharmaceutically acceptable carrier, diluent, or excipient.

10. A method of treating a disease, disorder, or condition comprising: administering to a human a compound according to claim 1, wherein the disease, disorder, or condition is selected from the group consisting of: cardiovascular disease, arteriosclerosis, hypertension, diabetes, diabetic-related complications, glomerular nephropathy, cerebral nerve degenerative diseases, Alzheimers disease, Parkinsons disease, ALS (amyotrophic lateral sclerosis), multiple sclerosis, asthma, chronic obstructive pulmonary disease, skin diseases, macular degeneration, cataracts, light retinopathy, retinopathy of prematurity, and cancer.

11. A method of increasing the activity of or the amount of HMOX1 in a subject comprising: administering to a subject a compound according to claim 1.
